(19) United States
(12) Patent Application Publication
(10) Pub. No.: US 2018/0353723 A1
(43) Pub. Date: Dec. 13, 2018

(54) Oxygen Administering System

(71) Applicant: Mildred Demeo, Park Hills, MO (US)

(72) Inventor: Mildred Demeo, Park Hills, MO (US)

(21) Appl. No.: 15/616,466

(22) Filed: Jun. 7, 2017

(51) Int. Cl. A61M 16/10; A61M 16/04; A61M 16/08

(52) U.S. Cl. CPC A61M 16/1005; A61M 16/0447; A61M 16/085; A61M 2230/432; A61M 2202/0208; A61M 2205/3303; A61M 2016/103

(57) ABSTRACT

A oxygen administering system for coupling an oxygen source and a CO2 monitor to a laryngeal mask airway includes a laryngeal mask airway that is inserted into a patient undergoing general anesthesia. An oxygen tube is fluidly coupled to an oxygen source to deliver gaseous oxygen and a CO2 monitor is provided to detect and monitor a flow of carbon dioxide. A coupler is selectively and fluidly coupled to the laryngeal mask airway. The coupler is selectively and fluidly coupled to the oxygen source to direct the gaseous oxygen into the laryngeal mask airway. Moreover, the coupler is in fluid communication with the CO2 monitor to monitor a flow of carbon dioxide that is exhaled through the laryngeal mask airway.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to oxygen devices and more particularly pertains to a new oxygen device for coupling an oxygen source and a CO2 monitor to a laryngeal mask airway.

### BRIEF SUMMARY OF THE INVENTION

An embodiment of the disclosure meets the needs presented above by generally comprising a laryngeal mask airway that is inserted into a patient undergoing general anesthesia. An oxygen tube is fluidly coupled to an oxygen source to deliver gaseous oxygen and a CO2 monitor is provided to detect and monitor a flow of carbon dioxide. A coupler is selectively and fluidly coupled to the laryngeal mask airway. The coupler is selectively and fluidly coupled to the oxygen source to direct the gaseous oxygen into the laryngeal mask airway. Moreover, the coupler is in fluid communication with the CO2 monitor to monitor a flow of carbon dioxide that is exhaled through the laryngeal mask airway.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a coupler of an oxygen administering system according to an embodiment of the disclosure.

FIG. 2 is a bottom view of a coupler of an embodiment of the disclosure.

FIG. 3 is a top phantom view of a coupler of an embodiment of the disclosure.

FIG. 4 is a left side view of a coupler of an embodiment of the disclosure.

FIG. 5 is a perspective in-use view of an embodiment of the disclosure.

### DETAILED DESCRIPTION OF THE INVENTION

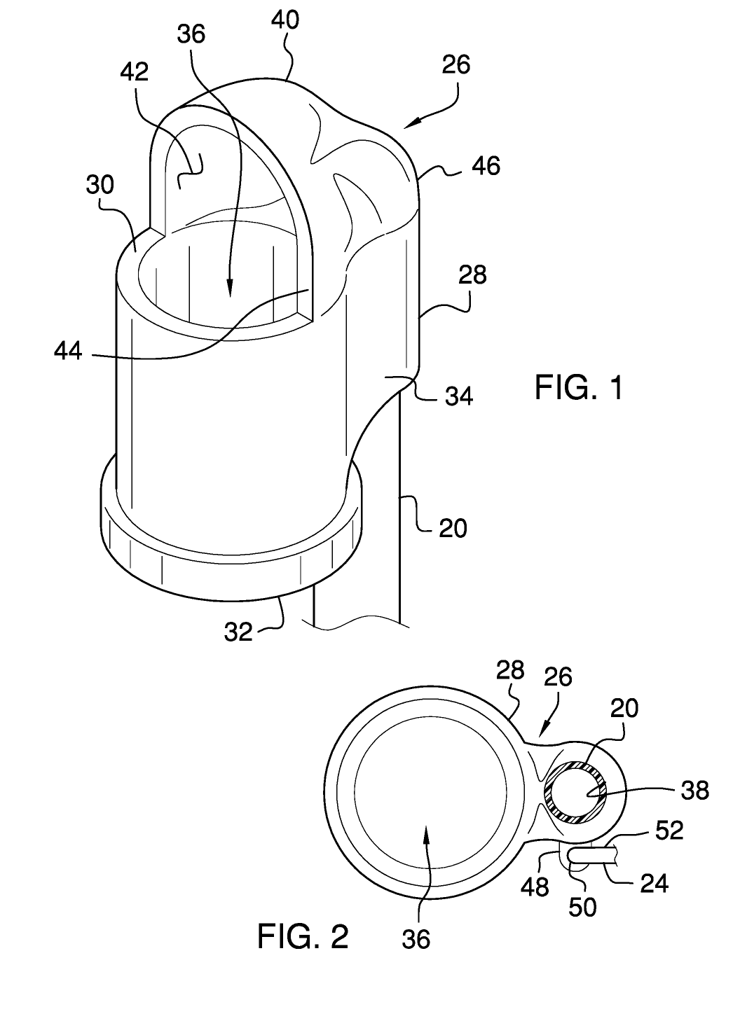

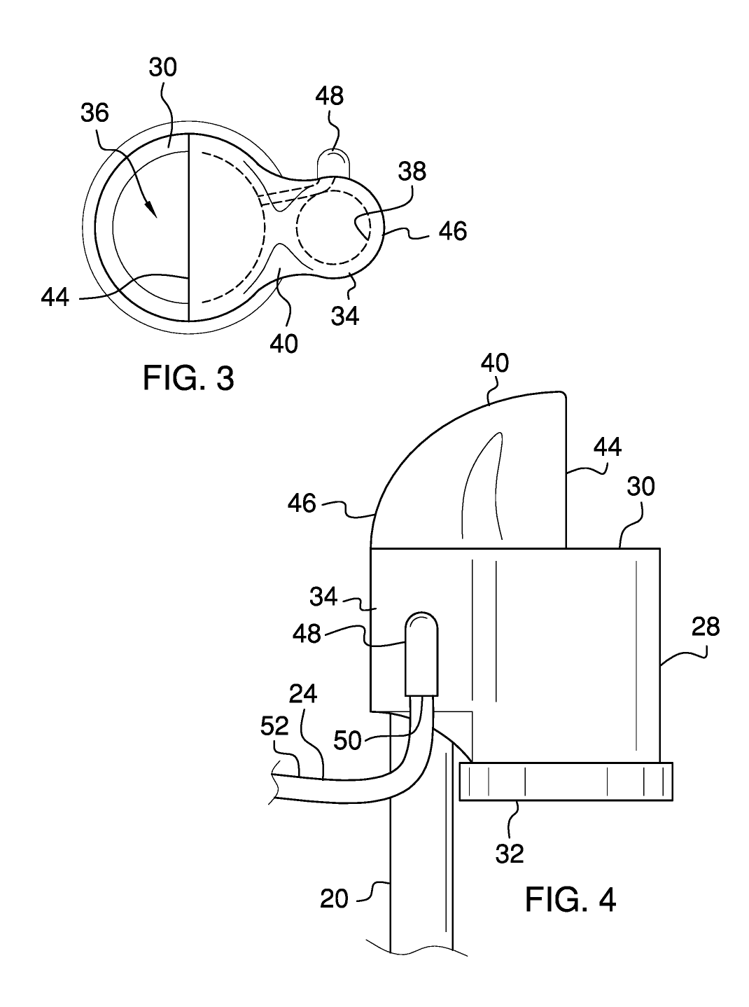

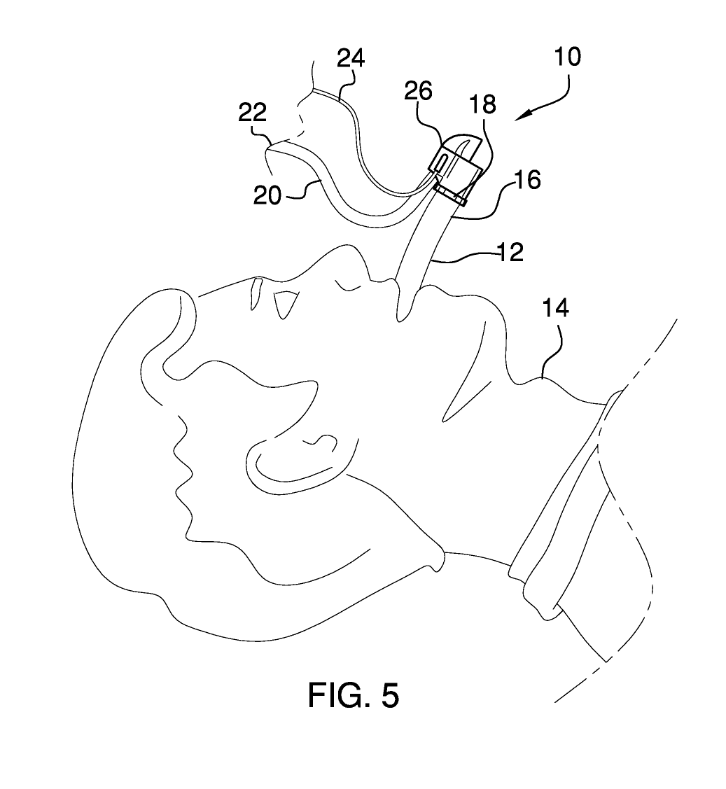

With reference now to the drawings, and in particular to FIGS. 1 through 5 thereof, a new oxygen device embodying the principles and concepts of an embodiment of the disclosure and generally designated by the reference numeral 10 will be described.

As best illustrated in FIGS. 1 through 5, the oxygen administering system 10 generally comprises a laryngeal mask airway 12 that may be inserted into a patient 14 undergoing general anesthesia. The laryngeal mask airway 12 includes a tube 16 and the laryngeal mask 12 may be any laryngeal mask 12 employed in a medical environment such as an emergency room, an ambulance, intensive care, recovery rooms and the like. The tube 16 has a first end 18.

An oxygen tube 20 is provided and the oxygen tube 20 is fluidly coupled to an oxygen source 22 to deliver gaseous oxygen. The oxygen source 22 may be a tank of compressed, gaseous oxygen or the like. A CO2 monitor 24 is provided to detect and monitor a flow of carbon dioxide. The CO2 monitor 24 may be an electronic CO2 monitor 24 commonly employed in a surgical environment.

A coupler 26 is provided and the coupler 26 is selectively and fluidly coupled to the laryngeal mask airway 12. The coupler 26 is selectively and fluidly coupled to the oxygen source 22 to direct the gaseous oxygen into the laryngeal mask airway 12. Additionally, the coupler 26 is in fluid communication with the CO2 monitor 24. In this way the CO2 monitor 24 may monitor a flow of carbon dioxide that is exhaled through the laryngeal mask airway 12.

The coupler 26 comprises a body 28 that has a first end 30, a second end 32 and an outer wall 34 extending therebetween. The body 28 has a first aperture 36 extending through the first end 30 and the second end 32. The first aperture 36 corresponding to the second end 32 insertably receives the first end 18 of the tube 16. Additionally, the body 28 has a second aperture 38 extending through the first end 30 and the second end 32.

The second aperture 38 corresponding to the second end 32 insertably receives the oxygen tube 20. In this way the second aperture 38 directs the gaseous oxygen outwardly through the first end 30 of the coupler. The outer wall 34 of the body 28 may undulate between the first 36 and second 38 apertures such that the body has a figure eight cross section taken along a line extending through the first 30 and second 32 ends of the body 28. Additionally, the first aperture 36 may have a diameter that is greater than a diameter of the second aperture 38.

A hood 40 is coupled to and extends away from the first end 18 of the body 28. The hood 40 has an inside surface 42 and the inside surface 42 is concavely arcuate with respect to the first end 18. The hood 40 has an open end 44 and a closed end 46 and hood 40 slopes upwardly between the closed end 46 and the open end 44. The closed end 46 surrounds the second aperture 38 to direct the gaseous oxygen toward the open end 44. Moreover, the open end 44 is aligned with the first aperture 36 corresponding to the second end 32 to facilitate the gaseous oxygen to mix with ambient air. Thus, the mixture of gaseous oxygen and ambient air is drawn through the tube 16 when the patient 14 inhales. The hood 40 additionally inhibit foreign objects from falling into the tube 16

A conduit 48 is coupled to the outer wall 34 of the body 28. The conduit 48 extends into the first aperture 36 such that the conduit 48 is in fluid communication with the first aperture 36. The conduit 48 has a primary end 50 and the primary end 50 is open to receive a hose 52 from the CO2 monitor 24. In this way the CO2 monitor 24 receives exhaled breath from the patient 14 for analysis. The coupler 26 may be a unitary structure with respect to the laryngeal mask airway 12 and the coupler 26 may be retro-fitted to existing laryngeal mask airways 12.

In use, the patient 14 is intubated with the laryngeal mask airway 12 when the patient 14 has undergone general anesthesia. The first end 18 of the tube 16 is plugged into the first aperture 36, the oxygen tube 20 is plugged into the second aperture 38 and the hose 52 from the CO2 monitor 24 is plugged into the primary end 50 of the conduit 48. The hood 40 directs the flow of gaseous oxygen toward the first aperture 36 to be inhaled by the patient 14. Additionally, the open end 44 of the hood 40 facilitates the gaseous oxygen to be mixed with ambient air. The patient 14 exhales through the first aperture 36 and the CO2 monitor 24 monitors the exhaled air. The coupler 26 is discarded when the laryngeal mask airway 12 is removed from the patient 14.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An oxygen administering system comprising:
a laryngeal mask airway being configured to be inserted into a patient undergoing general anesthesia;
an oxygen tube being fluidly coupled to an oxygen source wherein said oxygen tube is configured to deliver gaseous oxygen;
a CO2 monitor being configured to detect and monitor a flow of carbon dioxide; and
a coupler being selectively and fluidly coupled to said laryngeal mask airway, said coupler being selectively and fluidly coupled to said oxygen source wherein said coupler is configured to direct the gaseous oxygen into said laryngeal mask airway, said coupler being in fluid communication with said CO2 monitor wherein said CO2 monitor is configured to monitor a flow of carbon dioxide being exhaled through said laryngeal mask airway.

2. The system according to claim 1, wherein:
said laryngeal mask airway including a tube, said tube having a first end; and
said coupler comprises a body having a first end, a second end and an outer wall extending therebetween, said body having a first aperture extending through said first end and said second end, said first aperture corresponding to said second end insertably receiving said first end of said tube, said body having a second aperture extending through said first end and said second end, said second aperture corresponding to said second end insertably receiving said oxygen tube wherein said second aperture is configured to direct the gaseous oxygen outwardly through said first end.

3. The system according to claim 2, further comprising a hood being coupled to and extending away from said first end of said body, said hood having an inside surface, said inside surface being concavely arcuate with respect to said first end, said hood having an open end and a closed end, said closed end surrounding said second aperture wherein said inside surface of said hood is configured to direct the gaseous oxygen toward said first aperture corresponding to said second end.

4. The system according to claim 3, wherein said open end is aligned with said first aperture corresponding to said second end wherein said open end is configured to facilitate the gaseous oxygen to mix with ambient air thereby facilitating the mixture of gaseous oxygen and ambient air to be drawn through said tube when the patient inhales, said hood being configured to inhibit foreign objects from falling into said tube.

5. The system according to claim 2, further comprising a conduit being coupled to said outer wall of said body, said conduit extending into said first aperture such that said conduit is in fluid communication with said first aperture, said conduit having a primary end, said primary end being open to receive a hose from said CO2monitor wherein said CO2 monitor is configured to receive exhaled breath from the patient.

6. An oxygen administering system comprising:
a laryngeal mask airway being configured to be inserted into a patient undergoing general anesthesia, said laryngeal mask airway including a tube, said tube having a first end;
an oxygen tube being fluidly coupled to an oxygen source wherein said oxygen tube is configured to deliver gaseous oxygen;
a CO2 monitor being configured to detect and monitor a flow of carbon dioxide; and
a coupler being selectively and fluidly coupled to said laryngeal mask airway, said coupler being selectively and fluidly coupled to said oxygen source wherein said coupler is configured to direct the gaseous oxygen into said laryngeal mask airway, said coupler being in fluid communication with said CO2 monitor wherein said CO2 monitor is configured to monitor a flow of carbon dioxide being exhaled through said laryngeal mask airway, said coupler comprising:
a body having a first end, a second end and an outer wall extending therebetween, said body having a first aperture extending through said first end and said second end, said first aperture corresponding to said second end insertably receiving said first end of said tube, said body having a second aperture extending through said first end and said second end, said second aperture corresponding to said second end insertably receiving said oxygen tube wherein said second aperture is configured to direct the gaseous oxygen outwardly through said first end,
a hood being coupled to and extending away from said first end of said body, said hood having an inside surface, said inside surface being concavely arcuate with respect to said first end, said hood having an open end and a closed end, said closed end surrounding said second aperture wherein said inside surface of said hood is configured to direct the gaseous oxygen toward said first aperture corresponding to said second end, said open end being aligned with said first aperture corresponding to said second end wherein said open end is configured to facilitate the gaseous oxygen to mix with ambient air thereby facilitating the mixture of gaseous oxygen and ambient air to be drawn through said tube when the patient inhales, said hood being configured to inhibit foreign objects from falling into said tube, and
conduit being coupled to said outer wall of said body, said conduit extending into said first aperture such that said conduit is in fluid communication with said first aperture, said conduit having a primary end, said primary end being open to receive a hose from said CO2 monitor wherein said CO2 monitor is configured to receive exhaled breath from the patient.
